(19) United States
(12) Patent Application Publication
(10) Pub. No.: US 2019/0060103 A1
(43) Pub. Date: Feb. 28, 2019

(54) SEXUAL AID SYSTEM

(71) Applicant: Douglas Fielding, Langley (CA)

(72) Inventor: Douglas Fielding, Langley (CA)

(21) Appl. No.: 15/685,925

(22) Filed: Aug. 24, 2017

(51) Int. Cl. A61F 5/41

(52) U.S. Cl. CPC A61F 5/41; A61F 2005/414; A61F 2005/411

(57) ABSTRACT

A sexual aid system includes an elongated sleeve and a securer. The elongated sleeve is configured to encircle a shaft of a penis of a user leaving a head of the penis exposed. The securer is configured to secure the elongated sleeve to the user via attaching the securer to a rear of testicles and scrotum of the user. The elongated sleeve comprises a thickness and increases a girth of the penis of the user and provide additional pleasure to a male and female.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of sexual devices and more specifically relates to a penis enhancing device.

### 2. Description of Related Art

Individuals who experience lack and want for greater genital mass often become deeply embedded in a wide range of psycho-emotional inferiority complexes. This complex is becoming even more of a problem due to the “size matters” stigma that is being aggressively promoted in the media. This has had an immensely negative impact on the male perception of masculinity and thus his own sense of identity and self-worth. After child birth, a woman may lose the elasticity and vaginal tightness. This may lead to a decrease in pleasure during sexual intercourse. A suitable solution is desired.

U.S. Pub. No. 2012/0016189 to Bruce Victor Gioia relates to a male anatomical enhancement garment. The described male anatomical enhancement garment includes a male anatomical enhancement garment to enhance male genitalia with a base, a penis, a scrotum and testicles and a penis head that includes a base band to receive the genitalia that wraps around the base of the genitalia to secure the garment in place, a penis sheath to receive the penis that expands and contracts as the penis expands and contracts and a frontal aperture disposed on the end of the penis sheath that the inserted penis is inserted through. The garment also includes an internally embedded silicon polymer bead or attached elasticized band that is positioned behind the penis head after the penis is received through the front aperture and a pouch that contains the scrotum and testicles. There is also a glans cover that is provided to cover the penis head as well in another embodiment.

### BRIEF SUMMARY OF THE INVENTION

In view of the foregoing disadvantages inherent in the known sexual device art, the present disclosure provides a novel sexual aid system. The general purpose of the present disclosure, which will be described subsequently in greater detail, is to provide a penis girth enhancement garment.

A sexual aid system is disclosed herein. The sexual aid system includes an elongated sleeve and a securer. The elongated sleeve is configured to encircle a shaft of a penis of a user leaving a head of the penis exposed. The securer is configured to secure the elongated sleeve to the user via attaching the securer to a rear of testicles and scrotum of the user. The elongated sleeve comprises an open first-end and an open second-end. The open first-end allows the head of the penis to be exposed (being pushed therethrough). The securer is located proximate to the open second-end at a base of the penis. The securer may be permanently affixed to the proximate to the open second-end of the elongated sleeve. The elongated sleeve comprises a thickness and increases a relative girth of the penis of the user and increases pleasure for both parties during sexual intercourse.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a sexual aid system, constructed and operative according to the teachings of the present disclosure.

FIG. 1 is a perspective view of the sexual aid system during an ‘in-use’ condition, according to an embodiment of the disclosure.

FIG. 2 is a perspective view of the sexual aid system of FIG. 1, according to an embodiment of the present disclosure.

FIG. 3 is a perspective view of the sexual aid system of FIG. 1, according to an embodiment of the present disclosure.

FIG. 4 is a perspective view of the sexual aid system of FIG. 1, according to an embodiment of the present disclosure.

FIG. 5 is a perspective view of the sexual aid system of FIG. 1, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to sexual devices and more particularly to a sexual aid system as used to improve the pleasure and tightness during sexual intercourse for women who may have vaginal relaxation syndrome or other vaginal tightness issues.

Generally, the present invention is a sexual aid/implement granting additional girth to the penis during intercourse. The material is soft in nature, granting a natural experience for both parties. The shaft of the penis is encased in a sleeve, while the head of the penis remains exposed, granting pleasure to both the male and female parties. The device is secured to the penis using a loop of the same material attached behind the testicles and scrotum. The aim of the device is to provide additional pleasure to the female who might have experienced a loosening or increase in vaginal volume following child-birth or with age.

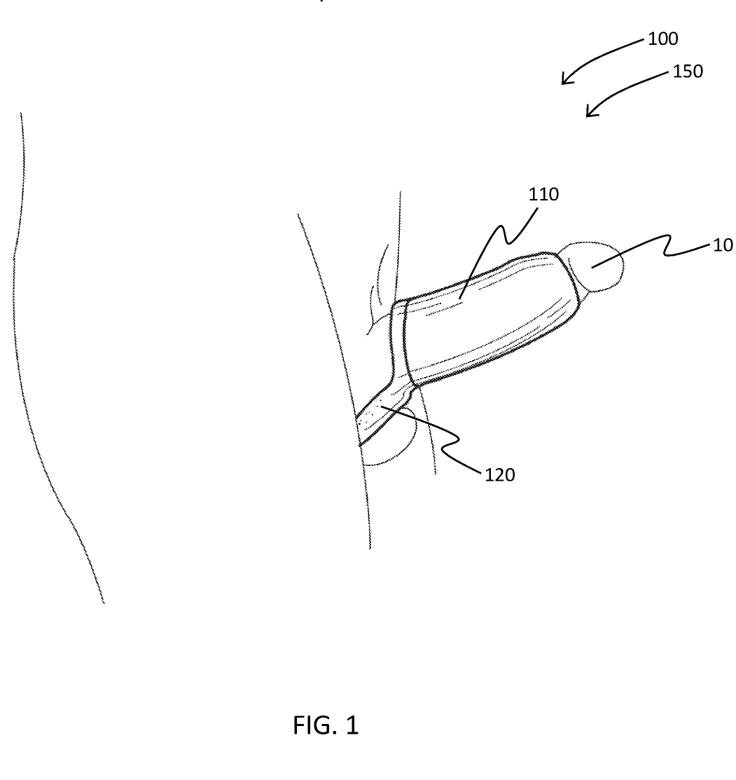

Referring now more specifically to the drawings by numerals of reference, there is shown in FIGS. 1-5, various views of a sexual aid system 100. FIG. 1 shows a sexual aid system 100 during an ‘in-use’ condition 150, according to an embodiment of the present disclosure. As illustrated, the sexual aid system 100 may include an elongated sleeve 110 and a securer 120. The elongated sleeve 110 is configured to encircle a shaft of a penis 10 of a user leaving a head of the penis 10 exposed. The securer 120 is configured to secure the elongated sleeve 110 to the user via attaching the securer 120 to a rear of testicles and scrotum of the user. The elongated sleeve 110 comprises a thickness and increases a ‘relative’ girth of the penis 10 of the user.

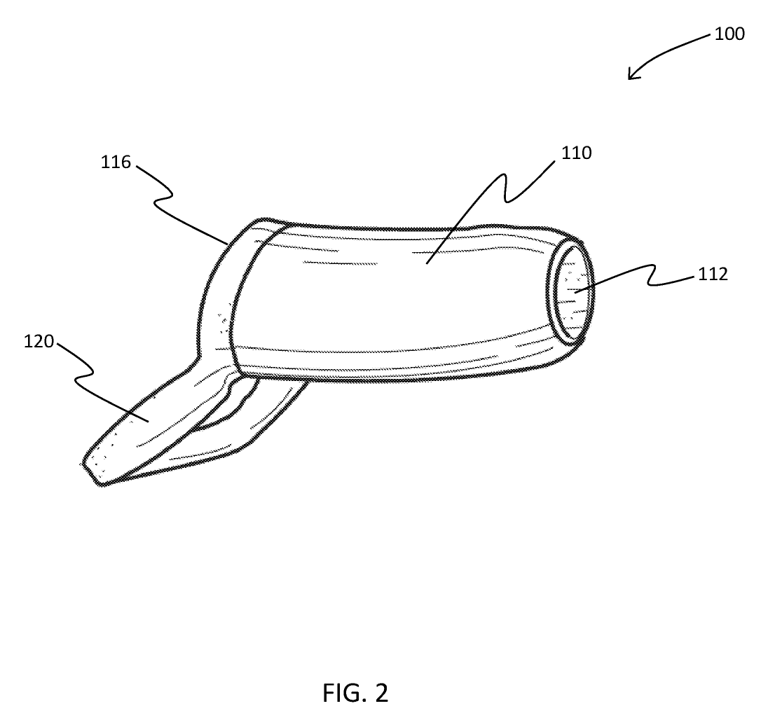

FIG. 2 shows a perspective view of the sexual aid system 100 of FIG. 1, according to an embodiment of the present disclosure. As above, the sexual aid system 100 may include the elongated sleeve 110 and the securer 120 in functional combination. The elongated sleeve 110 comprises an open first-end 112 and an open second-end 116. The open first-end 112 allows the head of the penis 10 to be exposed, as shown. The securer 120 is located proximate to the open second-end 116 at a base of the penis 10. The securer 120 may be permanently affixed to the proximate to the open second-end 116 of the elongated sleeve 110.

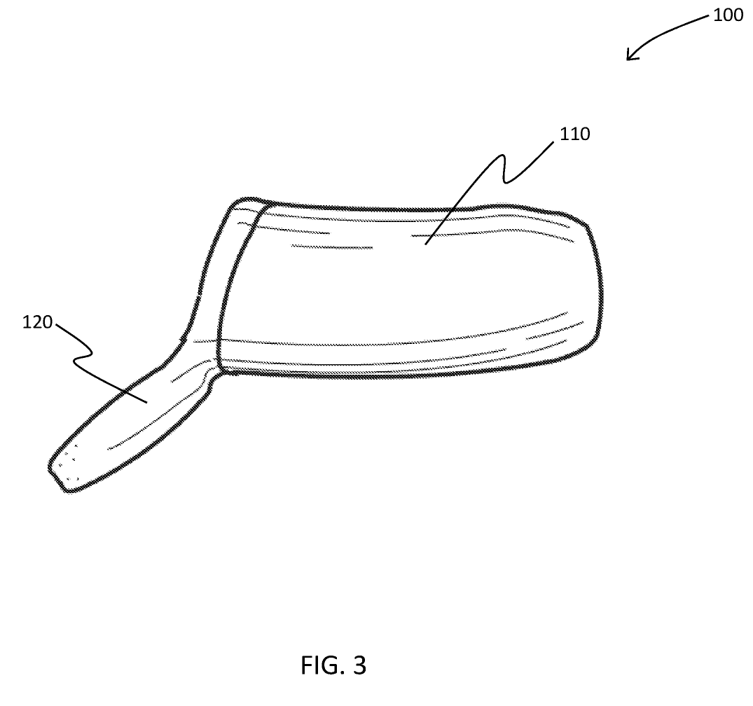

FIG. 3 shows a perspective view of the sexual aid system 100 of FIG. 1, according to an embodiment of the present disclosure. As above, the sexual aid system 100 may include the elongated sleeve 110 and the securer 120 suitable for increasing a girth of the penis 10 of the user. The securer 120 may comprise a circular profile creating a flexible loop fastener. The flexible loop fastener is configured to wrap around the rear of the testicles and the scrotum of the user. The flexible loop fastener and the elongated sleeve 110 comprise a material suitable for expanding and contracting as the penis expands and contracts (caused by relative blood flow due to arousal). The elongated sleeve 110 is cylindrical and comprises a length approximate to a shaft-length of the penis 10 of the user. The elongated sleeve 110 comprises a smooth outer surface. The elongated sleeve 110 and the securer 120 are elasticized and comprise the same material.

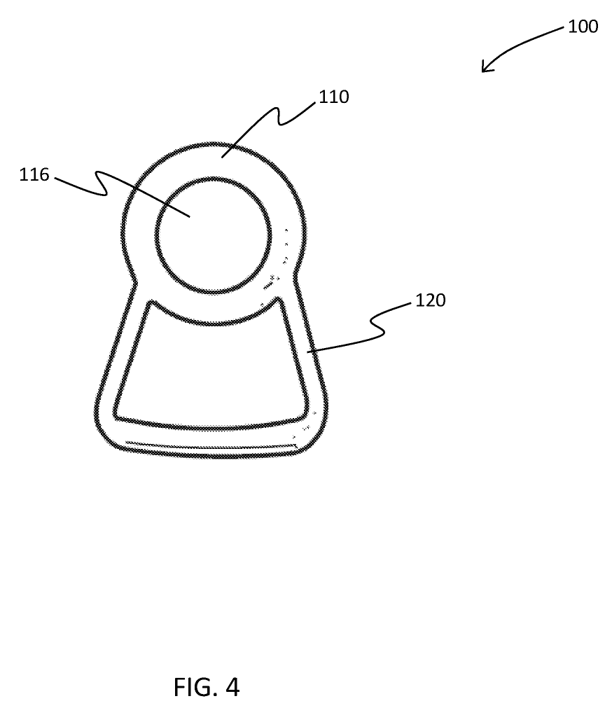

FIG. 4 shows a perspective view of the sexual aid system 100 of FIG. 1, according to an embodiment of the present disclosure. As above, the sexual aid system 100 may include the elongated sleeve 110 and the securer 120. The elongated sleeve 110 comprises a thickness and increases a girth of the penis 10 of the user. The elongated sleeve 110 is fitted to a penis 10 of a user. The thickness comprises at least a millimeter or other suitable thickness greater than 1 mm for creating additional thickness to the penis 10 for creating greater friction and tightness during sexual intercourse.

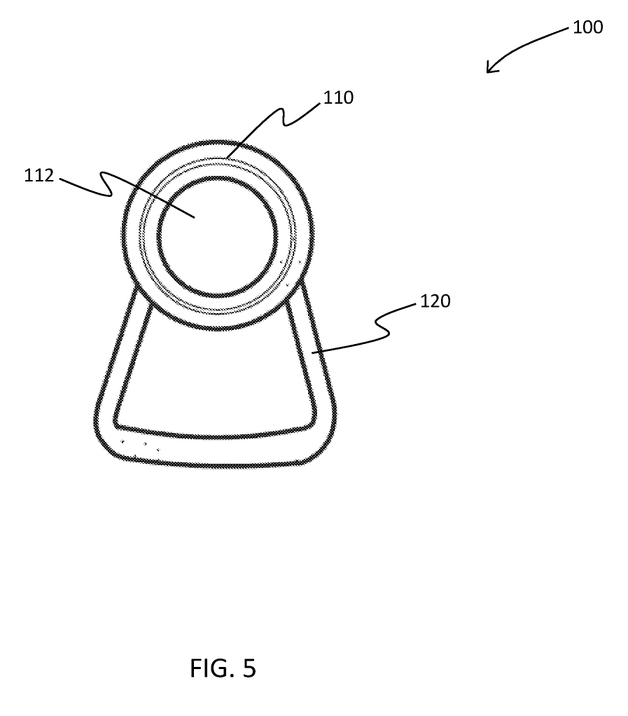

FIG. 5 shows a perspective view of the sexual aid system 100 of FIG. 1, according to an embodiment of the present disclosure. As above, the sexual aid system 100 may include the elongated sleeve 110 and the securer 120 providing a sexual aid granting additional girth to the penis 10 during intercourse.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A sexual aid system comprising:
an elongated sleeve; and
a securer;
wherein said elongated sleeve is configured to encircle a shaft of a penis of a user leaving a head of said penis exposed;
wherein said securer is configured to secure said elongated sleeve to said user via attaching said securer to a rear of testicles and scrotum of said user; and
wherein said elongated sleeve comprises a thickness and increases a girth of said penis of said user.

2. The sexual aid system of claim 1, wherein said elongated sleeve comprises an open first-end and an open second-end.

3. The sexual aid system of claim 2, wherein said open first-end allows said head of said penis to be exposed.

4. The sexual aid system of claim 2, wherein said securer is located proximate to said open second-end at a base of said penis.

5. The sexual aid system of claim 4, wherein said securer is permanently affixed to said proximate to said open second-end of said elongated sleeve.

6. The sexual aid system of claim 1, wherein said securer comprises a circular profile.

7. The sexual aid system of claim 1, wherein said securer comprises a flexible loop fastener.

8. The sexual aid system of claim 7, wherein said flexible loop fastener is configured to wrap around said rear of said testicles and said scrotum of said user.

9. The sexual aid system of claim 1, wherein said flexible loop fastener comprises a material suitable for expanding and contracting.

10. The sexual aid system of claim 9, wherein said elongated sleeve comprises said material suitable for expanding and contracting as said penis expands and contracts.

11. The sexual aid system of claim 1, wherein said elongated sleeve is cylindrical.

12. The sexual aid system of claim 11, wherein said elongated sleeve comprises a length approximate to a shaft-length of said penis of said user.

13. The sexual aid system of claim 12, wherein said elongated sleeve comprises a smooth outer surface.

14. The sexual aid system of claim 1, wherein said elongated sleeve and said securer are elasticized.

15. The sexual aid system of claim 1, wherein said elongated sleeve is fitted.

16. The sexual aid system of claim 1, wherein said thickness comprises at least a millimeter.

17. A sexual aid system comprising:
an elongated sleeve; and
a securer;
wherein said elongated sleeve is configured to encircle a shaft of a penis of a user leaving a head of said penis exposed;
wherein said elongated sleeve comprises an open first-end and an open second-end;
wherein said open first-end allows said head of said penis to be exposed;
wherein said securer is configured to secure said elongated sleeve to said user via attaching said securer to a rear of testicles and scrotum of said user;
wherein said securer is located proximate to said open second-end at a base of said penis;
wherein said securer is permanently affixed to said proximate to said open second-end of said elongated sleeve;
wherein said securer comprises a circular profile;
wherein said securer comprises a flexible loop fastener;
wherein said flexible loop fastener is configured to wrap around said rear of said testicles and said scrotum of said user;
wherein said flexible loop fastener comprises a material suitable for expanding and contracting;
wherein said elongated sleeve comprises said material suitable for expanding and contracting as said penis expands and contracts;
wherein said elongated sleeve is cylindrical;
wherein said elongated sleeve comprises a length approximate to a shaft-length of said penis of said user;
wherein said elongated sleeve comprises a smooth outer surface;
wherein said elongated sleeve and said securer are elasticized;
wherein said elongated sleeve is fitted;
wherein said elongated sleeve comprises a thickness and increases a girth of said penis of said user; and
wherein said thickness comprises at least a millimeter.
